(19) United States
(12) Patent Application Publication
(10) Pub. No.: US 2026/0264076 A1
(43) Pub. Date: Sep. 10, 2026

(54) Multiplexed Capillary-Flow Immunoassay And Methods of Use Thereof

(71) Applicant: Colorado State University Research Foundation, Fort Collins, CO (US)

(72) Inventors: Charles S. Henry, Fort Collins, CO (US); Jeremy Link, Fort Collins, CO (US)

(21) Appl. No.: 19/165,576

(22) PCT Filed: Mar. 15, 2024
(86) PCT No.: PCT/US2024/020200

§ 371 Date: Sep. 15, 2025

Related U.S. Application Data
(60) Provisional application No. 63/452,221, filed on Mar. 15, 2023.
(60) Provisional application No. 63/531,890, filed on Aug. 10, 2023.

(51) Int. Cl. B01L 3/00; G01N 33/543

(52) U.S. Cl. CPC B01L 3/50273; G01N 33/54387; B01L 2200/0684; B01L 2200/16; B01L 2300/0825; B01L 2400/0406; B01L 2400/0487

(57) ABSTRACT

A multiplexed capillary-flow immunoassay device configured for the simultaneous detection of multiple analytes, specifically, a testing assembly including a detection area, a fluid inlet, and a microfluidic network including at least two microfluidic pathways directed to distinct detection areas each configured to detect a different analyte.

### STATEMENT OF RELATED CASES

This International PCT Application claims the benefit of and priority to U.S. Provisional Application No. 63/452,221, filed Mar. 15, 2023, and U.S. Provisional Application No. 63/531,890, filed Aug. 10, 2023, both of which is incorporated herein by reference in their entirety.

### STATEMENT OF GOVERNMENT INTEREST

This invention was made with government support under grant number U01 HL152405 awarded by the National Institutes of Health. The government retains certain rights in this invention.

### TECHNICAL FIELD

The present technology is directed to the field of microfluidic diagnostic devices, and specifically a multiplex capillary flow device for the detection of multiple analytes from a single sample.

### BACKGROUND

The results of medical testing influences accurate diagnosis, prognosis, treatment, and timely discharge of patients from a medical facility. It follows that early and affordable diagnosis is a crucial part of the treatment process. The sooner a patient with chronic illnesses like cancer or Alzheimer's can be diagnosed, the more effectively the diseases can be slowed, halted, or prevented. Rapid testing for communicable diseases is also crucial for patient outcome. The need for reliable testing has been a theme for decades, and the world seems to get a grim reminder of this need every few decades with new or reemerging infectious diseases. In recent history, HIV/AIDS in the 1980's to present, SARS-COV in the early 2000s, H1N1 in the 2009, and SARS-CoV-2 in early 2019 have posed great stress to diagnostic infrastructure in place.

In both cases, communicable or non-communicable, diagnostics that can detect multiple targets are preferred to systems that can only detect one. For example, many assays have been developed to detect human C-reactive protein (CRP) to simplify heart failure diagnostic testing. Although levels increase after cardiac remodeling and fibrosis, CRP is also elevated from any inflammation, renal failure, liver disease, cancer, and/or in patients with COVID-19. Therefore, using CRP as the only target to monitor a patients risk of heart failure is ineffective. A more robust diagnostic tool would screen for CRP in conjunction with a panel of other markers. Similarly, being able to screen for and rule out other communicable diseases when a patient presents symptoms common to a variety of illnesses is important.

A diagnostic that can distinguish between two different marks indicative of a disease of condition, such SARS-COV-2 and the typical influenza would be a contemporaneous application. This example, SARS-COV-2 and the typical influenza highlights the need for fast and effective multiplexed assay. Specifically, both conditions share many of the same symptoms (fever, muscle soreness, cough, headaches, etc.), making it difficult to distinguish between the two solely on the symptoms a patient presents. Having a single diagnostic test that could differentiate between the two (or more) would have significant diagnostic and therapeutic advantages. In another example, elevated levels of CRP have been found in patients infected with SARS-COV-2. Subsequently, it has been suggested that elevated levels of CRP could be used as a predictor for the severity of a patient's condition. As a result, being able to detect a SARS-COV-2 infection and quantify CRP levels simultaneously would give more detailed information to a patient or health care worker interpreting the results. In yet another example, hormones control much of biological function and change as we age in response to menopause, etc. However, there is not a single hormone that accurately predicts changes and thus measuring multiple hormone analytes at once such as estradiol and testosterone can provide useful information in treating natural aging processes.

One of the most common ways to detect biological targets is enzyme-linked immunosorbent assays (ELISAs). Laboratory-based ELISAs are performed in 96-well microtiter plates. In this format, wells can be coated with a variety of capture proteins or antibodies. This spatial separation makes them ideal tools for multiplexing biological assays. Unfortunately, ELISAs are time consuming, requiring multiple pipetting steps and trained personnel to perform. These drawbacks hinder the ability for regular testing to occur, making it difficult to diagnose and treat patients. Rapid, onsite or point of care (POC) testing is especially important in rural and resource limited setting where a patient may have difficulty traveling to and from medical facilities.

Indeed, POC testing is an attractive alternative to traditional medical diagnostics in many ways. Reliable POC tests allow for faster time-to-results, more affordable and regular testing, and the opportunity for home testing and personalized medicine. Lateral flow assays (LFAs) have been a popular solution to POC diagnostics. LFAs consist of a series of membranes containing different reagents required for a sandwich immunoassay to occur from a single sample addition with a gold nanoparticle-based readout. LFAs can be used to assess environmental contaminants, food safety, and disease biomarkers. There has also been considerable effort in developing multiplexed LFA devices. The simplest approach to multiplex LFAs (xLFAs) includes the step of adding multiple test lines to a single test strip. This allows for multiple targets to be captured and screened in a single strip, but spatial constraints limit the number of analytes that can be targeted. Additionally, multiple capture and detection antibodies on the same test strip increase the chances of cross-reactivity and false positives from these reagents. Mathematical modeling has shown that test lines should be at least 2 mm apart for good detection. Extending the length of the test strip membrane could increase room for additional test lines, but assay time would increase exponentially according to the Lucas-Washburn equation.

Microarrays of test spots offer another alternative for increasing the number of detection zones, but spatial resolution and cross-reactivity between signal and capture antibodies still pose issues. Spatially separating multiple test strip membranes reduces the number of test line/targets per membrane. Successful devices have been designed where 10 individual LFA test strips radiate around a single sample inlet. This removes the possibility of cross-reactivity between antibody pairs and spatially separates test lines, but increases the total sample volume required to operate. Another example of multiple test strips on a single device comes from Han et. al. (39) which describes stacked paper channels controls sample and enhancer delivery to two separate test strips with three total test lines. This device sequentially delivers gold nanoparticle conjugated signal antibodies followed by an enhancer solution. However, the delivery method does not allow for washing between these steps, a highly desirable step for other applications like paper-based ELISAs.

Paper-based ELISA has been used for more than a decade in various ways. The simplest version of a paper-based ELISA replaces a 96 well plate with wax patterned paper microwells. While using paper may cut costs and improve portability, the manual pipetting steps required for this type of paper-based ELISA are still tedious. Several attempts have been made to simplify or automate the sample addition, washing, and regent delivery steps associated with an ELISA with the goal of making them more user friendly. While these examples are simpler than traditional ELISAs, they still require multiple steps after sample addition, disqualifying them as an ideal POC device. In another example, Henry et al., in PCT/US2021/071050 group has developed multiple automated, capillary driven immunoassay devices that fully automate sample delivery, washing, and reagent delivery steps to test strips, but multiplexing on these devices has yet to be shown.

As can be seen by the foregoing, there exists a long-felt need for a simple, and cost-effective multiplexed ELISA on a POC-style device that can accurately detect multiple analytes from a single sample.

### SUMMARY OF THE INVENTION

In one aspect, the technology is directed to a multiplexed capillary flow immunoassay having novel flow characteristics allowing multiplexed detection and analysis of multiple analytes in a single sample. In a preferred aspect, the device includes a microfluidic diagnostic device configured to transport a sample, reagents, and wash buffer to two separate test lines simultaneously with only a single end-user step.

In one preferred aspect, multiplex assay devices according to this disclosure may include a microfluidic network having two or more microfluidic pathways that in communication preferably with a single sample inlet, each having two or more dried reagents, which may be stored on pads within the microfluidic network or otherwise disposed within the microfluidic network. In certain embodiments, a first of the dried reagents may be an enzyme or nanozyme label while a second of the dried reagents may be a substrate. The microfluidic network is generally configured such that introduction of a sample, substantially fills the microfluidic network. The sample, which may or may not contain one or more analytes to be tested, may be transported to the detection zone by capillary action, while an additional portion of the sample, preferably containing a sample diluted in a buffer, is also directed to the detection zone thereby washing away excess sample that may interfere with the remaining assay. Following introduction of the fluid, dried reagent stored within the microfluidic network may be rehydrated by the fluid and generally permitted to flow toward the detection zone. In a two-reagent configuration, the pressure differential or other parameter impacting flow through the microfluidic pathways is such that rehydrated reagent from the first reagent pad, preferably an enzyme label, such as an enzyme labeled detection antibody arrives at the detection zone before rehydrated reagent from the second reagent pad. As a result, target analyte captured in the detection zone may capture the rehydrated reagent form the first reagent pad.

Following delivery of the first reagent, the second reagent, preferably a rehydrated substrate may be delivered to the detection zone. When the rehydrated second reagent reaches the detection zone, the rehydrated second may react with the first reagent, producing a visible color change or similar effect or reaction. As noted above, each sequential step described above can be accomplished following a single sample injection in parallel through adjacent microfluidic pathways. However, as described below, communication between each parallel pathways can be restriction through the targeted generation of an air bubble barrier, or other mechanical blocking components, positioned between adjacent microfluidic pathways blocking communication between them. In certain implementations, after flow is substantially complete in the device, the color or chemical change may generally be detectable with the naked eye for qualitative detection, imaged (e.g., using a smartphone camera) for quantitative information, or otherwise interpreted. One preferred aspect provides a novel automated, multiplexed ELISA on a POC-style device. Using hollow microfluidic channels, spatially separated test strips, and conjugate release pads, the device of the invention operates in a single sample addition step and outputs visually detectable signal in under 15 min. In a preferred aspect, the multiplexed assay device can rapidly detect two or more target analytes from a single sample with high sensitivity. For example, in one proof of concept example, the device of the invention demonstrated the simultaneous detection of HA from H1N1 in with a limit of detection (LOD) of 840 pg/mL, and N-protein from SARS-COV-2 with an LOD of 133 pg/mL when spiked in running buffer. While this device was designed to determine between a SARS-COV-2 or H1N1 infection, the device can also be used for simultaneous detection of two targets. In another example, N-protein and HA were detected simultaneously, and inactivated SARS-COV-2 and CRP were detected simultaneously, further demonstrating the utility of the device and methods of the invention to detect multiple target analytes simultaneously from a single sample.

Additional aspects of the invention may become evident based on the specification and claims presented below.

### BRIEF DESCRIPTION OF THE FIGURES

FIG. 1A-C: (A) Schematic with dimensions for each layer of the multiplexed Capillary-Driven Immunoassay (CaDI). (B) Order in which the 5 layers are assembled. (C) Location of reagent pads and nitrocellulose (NC) membranes containing tetramethylbenzidine (TMB), secondary antibody-Horse radish peroxidase (2° Ab-HRP) for each assay, and NC with capture and control antibodies for each assay.

FIG. 2A-B: (A) The single NC CaDI device was used to optimize conditions for the H1N1 assay. (B) TMB volume was held at 30 μL while the anti-hemagglutinin (anti-HA) 2° Ab-HRP concentration was varied. Following that, the anti-HA 2° Ab-HRP was held at 40 μg/mL while the TMB volume dried on the TMB pad was varied.

FIG. 3A-B: (A) The multiplex CaDI device function is broken down into 5 distinct key points. Image 1 depicts the device before sample is added. Image 2 is immediately after sample is added. Sample and buffer fill the channels (direction of flow is depicted with gray arrows), and dried reagents are rehydrated. Target present flows through NC membranes and binds to the corresponding capture antibodies. In image 3, 2° Ab-HRP for each side of the device is delivered to its respective test strip. In image 4, excess 2° Ab-HRP is washed from each test strip. Lastly, in image 5, TMB is delivered to each test/control line, reacting with H2O2 in the buffer and HRP on the NC if present. (B) Images focused on the NC are accompanied by a molecular level schematic for each crucial step outlined in A.

FIG. 4A-B: Dose response curves were run for both targets separately, and representative images are shown for selected points showing the test line (TL) and control line (CL). (A) SARS-COV-2 N-protein dose response curve was run while holding HA concentration at 0. The N-protein signal was fitted to a 4PL (R2=0.98). (B) H1N1 HA dose response was run holding the N-protein concentration at 0. The signal for HA was fit to a 4 PL fit (R2=0.97).

FIG. 5: Devices were run with 5 ng/ml and 100 ng/ml of both HA and N-protein in running buffer simultaneously.

FIG. 6: Devices were run with one side containing reagents and test strip for the N-protein immunoassay, and the other side with reagents for the CRP assay. Blank devices were run with neither target present as a control, and positive devices were run in triplicate with running buffer containing both 1100 PFU/mL SARS-COV-2 virus and 100 ng/ml CRP. Representative images for each assay are shown.

FIG. 7A-C: (A) Devices were run with 5 ng/ml and 100 ng/mL of both HA and N-protein in running buffer simultaneously. (B) The N-protein data from 5A is compared to the signal intensity in FIG. 4A when no HA is present in the running buffer. (C) The HA data from 5A is compared to the signal intensity from FIG. 4B where no N-protein is present in running buffer.

FIG. 8: shows an exemplary multiplexed capillary-flow immunoassay device.

FIG. 9: shows a schematic illustration of an enzyme linked immunosorbent assay (ELISA).

FIG. 10: Devices were run with one side containing reagents and test strip for the N-protein immunoassay, and the other side with reagents for the CRP assay. The CRP concentration was varied while the N-protein concentration in sample was held at 50 ng/mL. Representative images for each assay are included showing the location the test line (TL) and control line (CL).

### DETAILED DESCRIPTION OF THE INVENTION

The invention now being generally described will be more readily understood by reference to the following examples, which are included merely for the purposes of illustration of certain embodiments of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

The present invention is direct capillary-driven microfluidic devices and, more specifically, systems, methods and devices for a multiplex assay device configured to detect a plurality of target analytes in a single sample. In general, the devices disclosed herein include a microfluidic network through which fluid is transported by capillary action and that automatically sequences the delivery of reagents and washes to a test/detection zone for two or more target analytes. Sequencing of the detection between the multiple analytes, as well as their physical separation so as to prevent cross-reactivity or other contamination is achieved, at least in part, by varying the geometry and/or other flow driving characteristics of the different pathways through the microfluidic network. For example, the microfluidic network may include separate microchannel pathways for the detection of a unique target analyte. In preferred embodiments, these separate microchannel pathways can have varying lengths between, for example a sample inlet and the detection zones for each analyte such that fluid transported along longer paths is delivered to the detection zone after fluid transported along shorter paths. In another example, the microfluidic network may include microfluidic network having microchannel pathways configured to form fluid barriers, for example through the formation of bubbles at central positions between channels, preventing the flow of fluids between separate analyte channels. Dried reagents may be disposed along certain paths of the microfluidic network such that the reagents may be rehydrated and delivered to the detection zone sequentially, for example through analyte specific detection pathways. The flow driving characteristics of the paths (e.g., the geometry of the paths) relative to each other may therefore be used to control the timing and sequencing of delivery of the rehydrated reagents. Flow through the devices and subsequent sequencing of reagent delivery to the detection zone is substantially automatic and generally requires only that a user provide each of a sample and buffer solution or a combined sample and buffer to the device, depending on the particular test to be conducted using the device.

Traditional enzyme linked immunosorbent assays (ELISAs) are used to detect a wide range of analytes with good sensitivity and specificity. These analytes include whole cells, proteins, antibodies, and small molecules, among other things. The high sensitivity of ELISAs is enabled by stringent washing steps to mitigate non-specific adsorption of non-targets and using a catalytic label such as an enzyme to amplify a signal, while the high specificity is enabled using a “sandwich” immunoassay that captures an analyte between two highly specific probes.

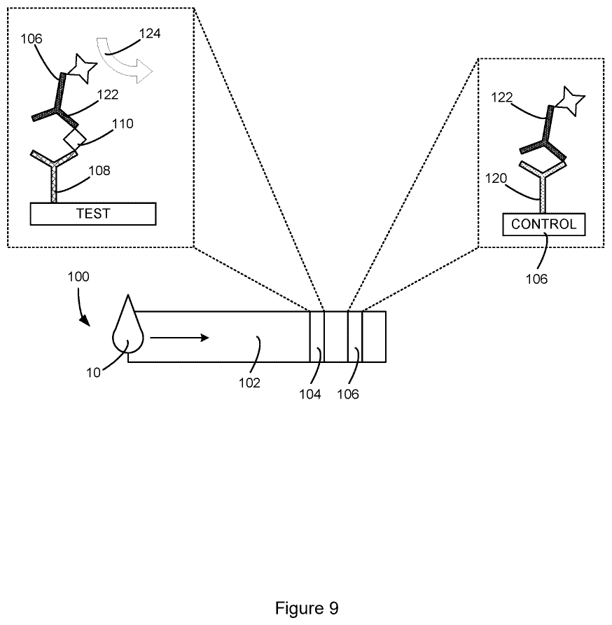

As shown generally in FIG. 9, a conventional ELISA system 100 includes a strip 102 that may have one or more detection zones or lines. In the specific example of this conventional ELISA system 100, the lines include a test line 104 and a control line 106. The strip 102 may be formed from nitrocellulose or a similar material such that, when a sample 10, is disposed on strip 102, sample 10 is wicked or otherwise transported to test line 104 and control line 106. A control line 106 may be optional but is generally included in ELISA systems to verify proper device functionality. In general, control line 106 includes an anti-label 120 adapted to bond with an enzyme label 122. Test line 104, on the other hand, includes a capture probe 108 configured to bond with a target analyte 110, to which enzyme label 122 bonds. Following delivery of target analyte 110 and enzyme label 122, an enzyme substrate 124 is provided that reacts with enzyme label 122 to produce a product and a corresponding colorimetric or other visual change of the corresponding line. Both enzymatic detection and the sandwich assay format require multiple washing steps and sequential delivery of reagents (e.g., the enzyme label and the substrate). These washing and reagent delivery steps yield robust analytical performance from ELISAs, but they are complicated to perform and relegate the assay to a centralized laboratory with expensive equipment and trained laboratory technicians. In many testing situations the time and financial resources needed for ELISAs are not available. There are more affordable point-of-care alternatives, but they often lack the analytical performance needed.

One of the most common alternatives to an ELISA is the lateral flow immunoassay. Lateral flow strips are inexpensive and easy to use in comparison to the ELISA. They generally require only that the end user provide a sample and the results are easy to interpret in most settings. The pregnancy test is the most widespread example of a lateral flow device. Although they are easy to use, lateral flow strips perform poorly compared to ELISAs, with well-documented reports of high false positive and negative rates and unreliable quantification. Substandard sensitivity and specificity in lateral flow assays stem from the inability to use enzymes as labels and sequentially wash and add reagents, which limits the scope of possible target analytes (e.g. disease biomarkers, antibodies, whole cells). These shortcomings are magnetized where the lateral flow strip is configured to detect more than one target analyte.

To address the foregoing issues, among others, this application introduces a system that can incorporate the advantages of a lateral flow assay, but with the same analytical capabilities of an ELISA, and in particular for the detection and quantification of more than one analyte in a sample. The system relies on a capillary driven immunoassay device to sequentially add and wash reagents and competing/non-target species from a distinct analyte test zones in fluid communication with a single sample inlet with minimal steps by an end user. The assay device generally includes a microfluidic network with channels that are specifically configured to provide at least two testing zones that are configured to all the sequential flow of reagents and washing fluids to spatially separated detection zones, each adapted to detect distinct target analytes. Operation of the assay device is substantially automatic, with an end user only being required to provide a sample and a buffer fluid or a sample/buffer combination. In certain implementations, flow through the assay device is achieved by capillary driven flow, which may be facilitated by hydrophilic channels of the microfluidic network and a passive pump mechanism, such as a paper/nitrocellulose pump. Notably, assay devices according to the present disclosure are capable of performing tests similar to conventional ELISAs, enabling simultaneous detection of a plurality of target analytes from a single fluid sample in at-home and other settings and by untrained users that were previously undetectable outside a centralized laboratory.

In certain implementations, the assay device is made of film sheets (e.g., transparency sheets, polyester film) and double-sided adhesive layers. Each layer may be laser cut or otherwise manufactured such that, when the layers are assembled by stacking and laminated, a microfluidic network is defined within the resulting laminated body.

The multiplexed microfluidic network of the invention includes multiple channels/paths, each configured to transport fluid by capillary flow from a sample inlet, sometimes also referred to as a fluid inlet through at least two to analyte zones to a testing area/detection zone. Certain of the channels may include analyte-specific dried reagents that are rehydrated by fluid transported through the channels for subsequent delivery to the testing area. Other channels may not include reagents such that fluid transported through such channels is delivered to the testing area without substantially altering the composition of the fluid. As a result, fluid transported through channels furnished with a dried or otherwise stabilized reagent may be used to deliver the reagent to the testing area.

Washes may be provided, for example, by fluid portions that do not include rehydrated reagent or that are provided by other paths/channels of the microfluidic network that do not include dried reagents. By varying the length, size, number, and similar characteristics of the channels of the microfluidic network and including other flow control mechanisms, delivery of fluid via different channels to the testing area/detection cone may be sequenced to detect multiple target analytes from a single sample. So, for example, delivery of reagents and washes may be alternated or otherwise sequenced to follow a particular testing protocol that may further be adapted to detect two or more distinct target analytes.

Dried reagents may be provided by conjugate release pads disposed along channels of the microfluidic network. Such pads may be formed, e.g., from glass fiber or nitrocellulose. Alternatively, dried reagent may be disposed along a given channel, e.g., by being dried onto a surface of the channel.

In certain preferred embodiment, the testing area may include a plurality of a nitrocellulose (NC) membranes connected to or otherwise in communication with the multiplexed microfluidic network, and preferably in fluid communication with a distinct analyte detection pathway. The nitrocellulose membranes may contain a capture probe specific to a target analyte. The capture probes may be striped onto the individual nitrocellulose membranes to form a test line for a specific target analyte. During operation, a detection probe, such as an enzyme-labeled detection probes which can include labeled antibodies or even aptamers specific to the target analytes may be delivered to their respective test line (e.g., by rehydrating a dried probes disposed in a channel of the microfluidic network) followed by subsequent delivery of a substrate (e.g., by rehydrating a dried antibody disposed in a channel of the microfluidic network) that reacts with the enzymatic label to produce a signal at the test line.

The assay device may include a specific sample inlet and related components for processing a sample having more than one analyte for testing, such as a filtration membrane for filtering the sample. When performing an assay based on whole blood, for example, the filtration membrane may be a plasma separation membrane and may be sealed over the sample inlet. In such cases, the assay device may further include a separate fluid/buffer inlet for introducing a fluid buffer to initiate subsequent testing of the sample. For less viscous or less complex sample matrices (e.g., nasal swab samples diluted in an extraction buffer) a single sample inlet/buffer inlet with no filtration membrane could be used. In such application, a separate membrane or similar element may not be necessary, and the sample may also supply the buffer for the rest of the assay. In general, however, the same reagent addition and washing steps may be accomplished with single-inlet assay device as with multi-inlet devices by configuring the microfluidic network accordingly.

Example embodiments discussed herein focus on non-competitive immunoassays; however, implementations of the present disclosure are not limited to such assays. Rather, this disclosure is intended to describe a more general assay device that may be used in a range of applications. Stated differently, this disclosure is intended to describe a general assay device capable of sequential delivery of reagents and washes to detect a plurality of distinct analytes, which may preferably be through a colorimetric or chemiluminescent detection assay. Although specific tests (including specific sample types, reagents, buffers, etc.) may be described, such tests should be considered illustrative only and non-limiting regarding other applications for the present disclosure. For example, while most examples discussed herein focus on testing of biological samples, such as one or more bodily fluids, assay devices according to this disclosure may be adapted for environmental or chemical testing (e.g., water testing).

As another example, while the examples disclosed herein generally focus on non-competitive immunoassays, assay devices according to the present disclosure may be adapted to perform competitive immunoassays. Competitive immunoassays are more commonly used for small molecules, like hormones, THC, and other small molecular weight molecules where an antibody pair is not available. In a competitive immunoassay, a capture antibody is deposited in a detection area (e.g., on a nitrocellulose strip). A detection reagent in the system would be a labeled version of the analyte (e.g., the analyte labelled with an enzyme, metal, nanoparticle, etc.). When the sample runs through the system with no analyte, the labeled detector analyte binds to the capture probe, which may preferably include a capture antibody or capture aptamer, and gives a signal, e.g., a color change at the test line. If analyte is present in the sample, it competes with the detector analyte binding at the detection area and reduces the resulting signal. Therefore, a reduction in signal is observed in the presence of specific analyte. So, for example, in implementations of the present disclosure, an assay device may be configured to deliver a capture antibody to a detection area followed by a sample, followed by a labeled version of a plurality of distinct analytes, with optional washes between each delivery.

More generally, while specific assay devices are described herein, such devices may be readily adapted for different applications by modifying the placement and type of reagents included in the assay device as required for the assay to be performed. For example, in one preferred embodiments, a multiplexed assay device of the invention may include a colorimetric assay, while in alternative embodiments, the multiplex assay device of the invention can be a chemiluminescent assay, and another embodiment where the multiplex assay of the invention can be used for electrochemical detection. All of the forgoing examples being accomplished with a multiplexed assay device configured to detect two or more distinct analytes from a single sample.

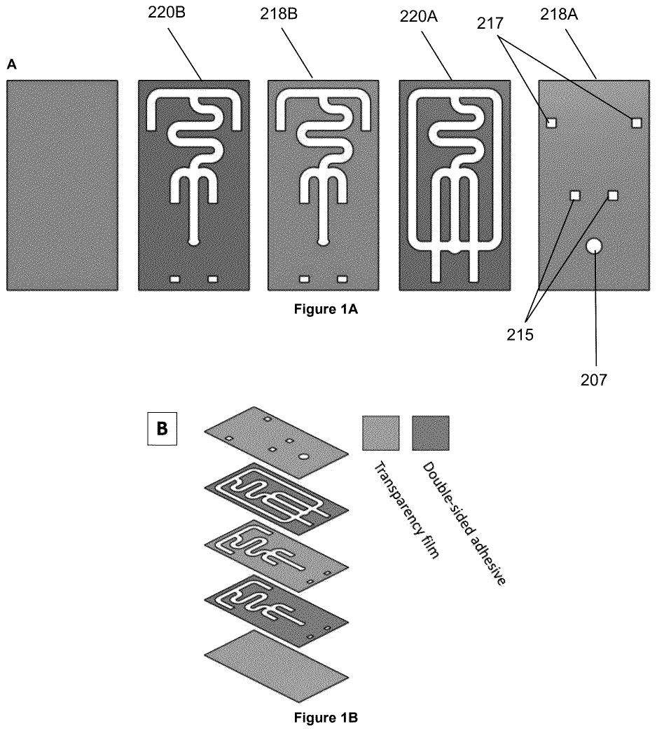

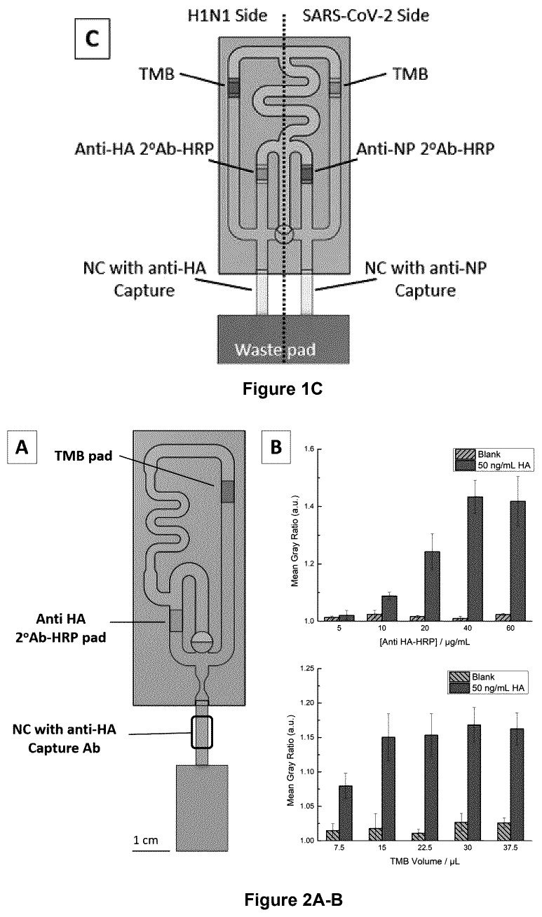

The multiplex assay device 200 of the invention (also referred to herein as a multiplexed assay device, or device, or device of the invention) includes a device body 202 defining a multiplexed microfluidic network 204 configured to detect at least two separate analytes. The multiplex assay device 200 includes a sample inlet 206 in communication with multiplexed microfluidic network 204. As shown in FIG. 1A, individual layered components of the multiplex assay device 200 is generally illustrated with certain layers of device body 202 selectively separated for better illustrating aspects of assay device 200 and, more specifically, microfluidic network 204. In a preferred embodiment, multiplexed assay device 200 comprises a microfluidic chip be formed from multiple layers including from alternating layers of film (e.g., film layers 218A, 218B and double-sided adhesive (e.g., double-sided adhesive layer 220A, 220B).

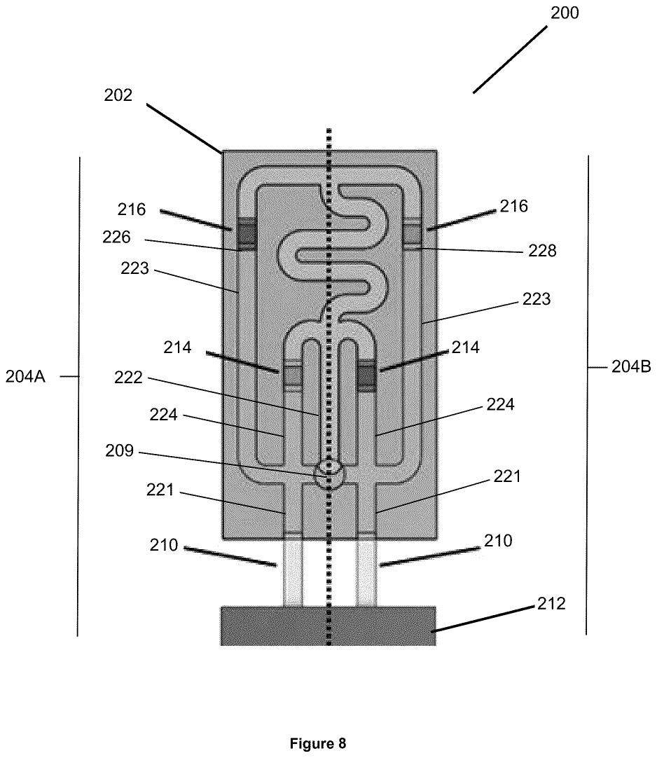

Alternatively, assay device 200 may be 3D printed or manufactured using any other suitable techniques. Again, as illustrated in FIG. 1A, film layer 118A, which corresponds to a topmost layer of the device 100, may define various openings. More specifically, film layer 718A includes each of an inlet opening 215 corresponding to sample inlet 206 (shown in FIG. 8) and each of a first set of vents 215 corresponding to dried enzyme label pads 214 dried enzyme label pads 214 of each microfluidic pathway 204a, 204b (shown in FIG. 8) and a second set of vents 217 corresponding to dried substrate pads 216 of each microfluidic pathway 204a, 204b (shown in FIG. 1). Notably, in some cases a dried enzyme label pads 214 may contain a labeled probe, which can be a non-enzyme probe, as well as a labeled antibody or aptamer probe. Such embodiment are specifically claimed herein. As noted above in the context of assay device 100, vents 215, 217 generally facilitate filling of device body 202 with fluid while preventing formation of air bubbles that may disrupt flow through microfluidic network 204, outside of areas within the network specifically designed for form air bubbles as barriers to prevent contact between rehydrated enzyme or substrate materials. In certain other embodiments, a sample may require processing as part of the testing process. In certain cases, embodiments, sample inlet 206 may include a filtration membrane or similar component for processing the sample. For example, when testing blood, a sample inlet 206 may include a plasma or similar membrane to separate blood components.

As shown in FIG. 8, the multiplexed microfluidic network 204 generally includes at a first and second microfluidic pathways 204a, 204b forming channels for transporting a fluid provided via sample inlet 206 to a pair of spatially separated test strips 210 (e.g., a colorimetric or chemiluminescent test strip). In general, the channels of each microfluidic pathways 204a, 204b are configured to detect a distinct target analyte in a fluid transported preferably by capillary action. Such transportation may be facilitated by forming device body 202 from or otherwise applying hydrophilic materials to surfaces of the channels of microfluidic network 204. Transportation may be further facilitated by a nitrocellulose or similar “wicking” substrate of test strip 210 alone or in combination with a passive pump 212, which may be in the form of a waste pad that also collects excess fluid. In alternative embodiments, a buffer inlet (not shown) can also be formed by the device body 202 and be in fluid communication with the microfluidic network 204 to allow introduction of a buffer into the microfluidic network 204.

The multiplex assay device 200 of the invention is generally configured to perform a test like a conventional enzyme linked immunosorbent assay (ELISA) to detect or identify between at least two target analytes in a sample. To facilitate such testing, assay device 200 includes dried reagents disposed along channels of microfluidic network 204. Specifically, dried reagents for the detection of a first target analyte are disposed along channels of a first microfluidic pathways 204a, while dried reagents for the detection of a second target analyte are disposed along channels of the second microfluidic pathways 204a. In the preferred embodiment shown in FIG. 8, the multiplex assay device 200 includes a dried enzyme label pad 214 and a dried substrate pad 216 disposed within each of the first and second microfluidic pathways 204a, 204b forming the microfluidic network 204. In other implementations, assay device 200 may be adapted to perform other assays by changing, adding, removing, or otherwise modifying the specific reagents included in the first and second microfluidic pathways 204a, 204b of the microfluidic network 204. Such modification may include adding additional paths of microfluidic network 204 for delivery of additional reagents, such as a third, fourth, fifth or even a sixth microfluidic pathway, each preferably configured to detect a distinct target analyte.

The configuration of the multiplex assay device 200 is such that a sample, detection probe and enzyme label, and substrate are each delivered to spatially separated test strips 210 sequentially with intervening washes. More specifically, a user of multiplex assay device 200 provides a sample via sample inlet 206, which is transported to through the first and second microfluidic pathways 204a, 204b of the microfluidic network 204 to a corresponding test strip 210. In this configuration, the fluid within the microfluidic network 204 rehydrates the reagents on dried enzyme label pad 214 and dried substrate pad 216 within the and enables transport of the reagents through their respective microfluidic pathways 204a, 204b to a test strip 210. In a preferred embodiment, a sample is introduced to device 200 through the sample inlet 206 and disperses through the microfluidic network 204. In this specific embodiment, a portion of the sample containing two or more distinct target analytes is directed to first and second microfluidic pathways 204a, 204b of the microfluidic network 204, where it rehydrates the dried enzyme label pad 214, and dried substrate pad 216, sometimes generally referred to as conjugate release pads.

Notably, the sequential reagent delivery and washing illustrated herein is facilitated by varying the geometries of flow paths through the microfluidic networks. For example, all other geometries being equal, fluid transported along a first path of a microfluidic network will arrive before fluid transported along a second path of the microfluidic network when the second path is longer. Accordingly, the first path may be used to deliver a first reagent to a test strip at a first time and the second path may be used to deliver a second reagent to the test strip thereafter. Similar results may be achieved by altering other geometric characteristics (e.g., surface area, convolutional characteristics, cross-section shape, etc.) of flow paths within the microfluidic network, forming flow paths from different materials, coating surfaces of the flow paths with different materials, and the like. For example, a first path may include a surface that is more hydrophilic then a surface of a second path to drive capillary flow. In such cases, fluid transported along the first path will generally arrive at a destination prior to fluid transported along the second path, all other things being equal. For example, a first path may include a convolutional shape, while a more straight line shape can facilitate transfer of the fluid more directly.

Again, referring to FIG. 8, the dried enzyme label pads 214 are positioned within their respective microfluidic pathways 204a, 204b relative to dried substrate pads 216 such that rehydrated enzyme label arrives at test strip 210. Accordingly, in at least certain embodiment, device body 202 may include a topmost layer (e.g., layer 218A) that substantially covers and contains microfluidic network 204 and components disposed therein, as generally illustrated in FIGS. 4 and 8. Notably, the topmost layer may include various openings for providing various functions. In addition to openings to permit introduction of samples and/or buffer fluid (e.g., a sample inlet opening 207 corresponding to sample inlet 206, the topmost layer may include vents or similar openings corresponding to each of dried enzyme label pad 214 and dried substrate pad 216. For example, device body 202 includes each of a first vent 215 for dried enzyme label pad 214 and a second vent 217 for dried substrate pad 216. As discussed below, such venting functions permit proper filling of assay device 200 with fluid, such as a buffer or other fluid, and generally preclude the formation of air bubbles that may negatively impact or disrupt flow through microfluidic network 204, except as described above.

Again, as shown in FIGS. 4 and 8, each of the first and second microfluidic pathways 204a, 204b are in fluid communication with a corresponding test strip 210 configured to receive the sample. Based on the configuration and distance between the sample inlet 206 and the corresponding test strips 210, the sample is separated and flows through each of the spatially separated strips 210. In this preferred embodiment, if one or more target analytes are present in the sample they will bind to their respective capture probes, in this case being a capture antibody, striped on its respective test strip 210. The remining fluid, being dispersed through the microfluidic network 204 are pulled through capillary forces generated by a passive pump 212 of the invention, in this case a waste pad and the test strips respectively. As the channels of the microfluidic network 204 are drained, an air bubble forms in the center channel 222 above the sample inlet 206. Because of this barrier formed by the bubble, the contents of dried enzyme label pads 214, preferably containing an enzyme label such as a secondary antibody directed to a target analyte as described herein, positioned in the adjacent first and microfluidic pathways 204a, 204b are prevented from flowing together and interacting within the microfluidic network 204 of the device and the opposite assay's test strip 200.

Again, referring to FIG. 8, the based on the configuration of the microfluidic network, the rehydrated contents of dried enzyme label pads 214, preferably containing an enzyme label, such as a secondary antibody are individually delivered to their respective test strips 210. As noted above, the enzyme label can react with the target analyte bound to the capture probe, preferably along one or more test or control lines. Subsequent to the delivery of the enzyme label, a quantity of fluid, preferably containing a portion of buffer is individually directed to the test strips 210 thereby washing them of excess enzyme label.

Lastly, as shown again in FIGS. 4 and 8, again, based on the configuration and lengths of the channels of the microfluidic network 204, a fluid from the sample inlet 106 rehydrates substrate from the dried substrate pad 216 in each of the microfluidic pathways 202a, 204b, which are then directed by capillary action to their respective test strips 210 as generally described herein. After delivery of rehydrated substrate, a certain time may be required to elapse before an observable change, such as a colorimetric or chemiluminescent change is observed at each of the test strips 210, indicating the result of the assay.

Notably, embodiments of the present disclosure are not limited to any specific enzyme label and the specific enzyme label used may vary based on the specific assay to be performed using multiplex assay device 200. For example, in at least certain embodiment, the enzyme label may be either of a biological enzyme or a non-biological enzyme (e.g., a nanozyme). In certain implementations, a sample may require processing as part of the testing process. In such cases, sample inlet may include a filtration membrane or similar component for processing the sample. For example, when testing blood, sample inlet 206 may include a plasma or similar membrane to separate blood components. In other embodiments, the sample may include a buffer, while in alternative embodiment a buffer can be introduced separately into the sample inlet or another buffer inlet (not shown)

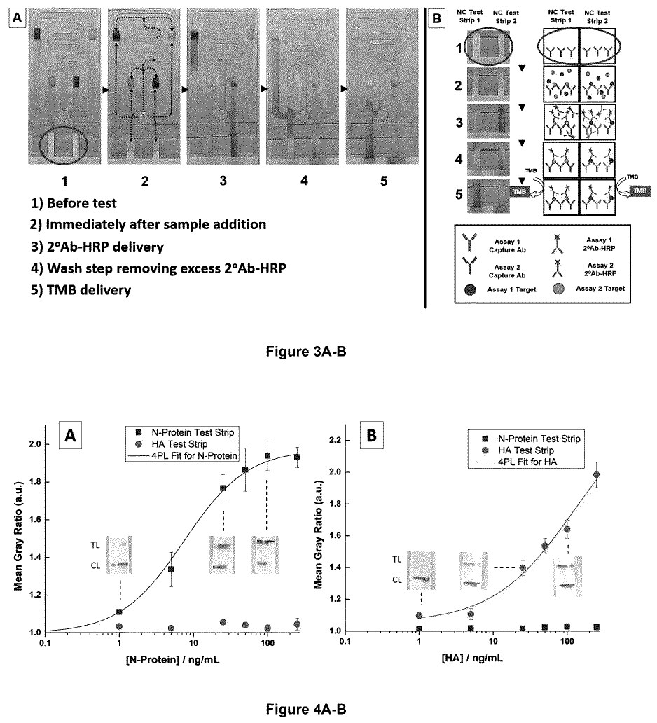

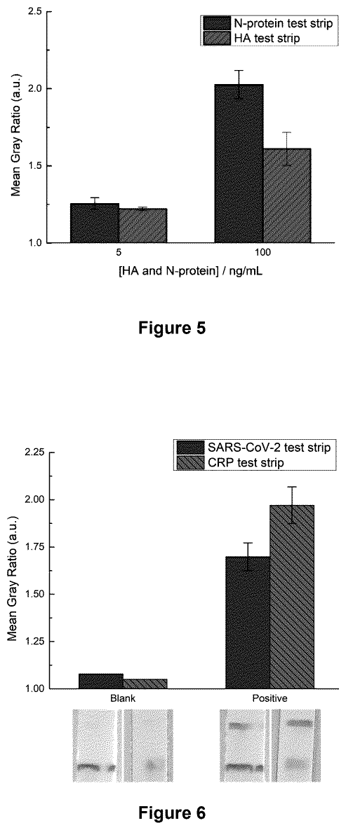

FIG. 3A-B illustrates the sequential operation of an exemplary multiplex assay device 200 of the invention. More specifically, each of images 1-5 include a photograph of multiplex assay device 100 positioned adjacent to a schematic illustration of test strip 210 indicating the status of the strip and any reagents/substances provided thereto. As noted below, food coloring was used in place of actual enzyme labels and reagents to better illustrate operation of assay device 200; nevertheless, the following example still refer to enzyme labels and substrates. Image 1 of FIG. 3A illustrates the multiplex assay device 200 in a pre-assay state. In the pre-assay state, fluid has not yet been provided to inlet 106 and distributed to the microfluidic network 204 and each of enzyme label pads 214 and dried substrate pads 216, disposed along their respective microfluidic pathways, include corresponding dried reagents. As illustrated in the detailed view of test strips 210, each test strip 210 may include a distinct capture probe (generally shown as capture probe 752a, and 752b adapted to bond with a distinct target analyte. As previously discussed, test strips 210 may include other capture probes for purposes of testing and validating operation of the multiplex assay 200.

FIG. 3A-B, image 2 illustrates an exemplary multiplex assay device 200 following addition of a test fluid to inlet 206. In general, the fluid is distributed from inlet 206 to different channels of microfluidic network 204 by capillary action. Based on the configuration of the microfluidic network 204 in relation to the fluid inlet 206, the fluid simultaneously fills the all channels 204a and 204b of the device (direction of flow is shown in gray arrows in FIG. 3A, image 2) and is transported to the test strips through corresponding outlet channels 221 where each analyte 250 can bind to its corresponding capture probes 752a, 752b, shown here as a capture antibody, stripped to each test strip 210. A portion of sample fluid is distributed through the first and second microfluidic pathways 204a, 204b of the microfluidic network 204 and contacts the first conjugate release pad along the inner channels 224 of the network. In this preferred embodiment, the sample fluid is distributed through the inner channels of the first and second microfluidic pathways 204a, 204b of the microfluidic network 204 contacting a dried enzyme label pad 214 positioned within each distinct channel. The distributed fluid initiates rehydration of the dried enzyme labels stored on dried enzyme label pads 214. Similarly, a portion of the fluid is distributed along the longer outer channels 223, as well as through an inner central channel 222 to contact a conjugate release pad that is positioned further from the sample inlet 124. In a preferred embodiment, this second conjugate release pad preferably includes a dried substrate pad 216, fluid contact of such, initiates rehydration of the dried substrate.

Referring now to FIG. 3A-B, image 3, following introduction of the sample fluid and substantial elapsed time such that a substantial portion of the rehydrated enzyme label 228 is transported, from dried enzyme label pad 214 to test strip 210. As further shown in image 3, a portion of rehydrated substrate 226 has simultaneously started moving toward test strip 210. Also, as illustrated in the detail view of test strip 210, at least a portion of rehydrated enzyme label 228 arriving at test strip 210 binds with target analyte 250 previously captured by the at test strip 210, thereby preparing test strip 210 for delivery of rehydrated substrate 226. As noted elsewhere, transport of rehydrated enzyme label 228 occurs in parallel through each microfluidic pathway 204a, 204b and is directed to separate test strips 210 configured to detect a specific target analyte 250.

Again, referring to FIG. 3A-B, image 3, during sequential delivery of enzyme labels from dried enzyme label pads 214, an excess of enzyme label may be delivered to their corresponding test strips 210. Accordingly, portion of fluid is directed through the individual microfluidic networks 204a, 204b to their corresponding test strips 210 which washes the strip after delivery of rehydrated enzyme label 228. More specifically, microfluidic network 204 is generally shaped and configured such that at least a portion of the test fluid is delivered to the test strips 210 after rehydrated enzyme label 228 but before rehydrated substrate 226. By doing so, excess of the enzyme labels can be removed from test strips 210 (as is shown in image 4, where the dye representing rehydrated enzyme label is washed from the test strips 210 before arrival of rehydrated substrate 226, generally improving the response of test strips 210.

FIG. 3A-B, image 5, illustrates multiplex assay device 200 during the sequential delivery of rehydrated substrate 226 through the microfluidic pathways 204a, 204b to their corresponding test strip 210. As shown in the image 5, delivery of rehydrated substrate 226 results in the production of a product, in this case comprising the reaction of the substrate 226 with the enzyme label 228 which may, in turn, cause an observable change in test strip 210, such as the appearance of a colored strip, or chemiluminescent signal within the detection zone. Notably, observable change in test strip 210, such as the appearance of a colored strip, or chemiluminescent signal within the detection zone, may be from the presence of targeted analyte or from signal produced on a control line, as described herein.

Notably, operation of any of the foregoing devices and others in accordance with the present disclosure is straightforward from the perspective of an end user and substantially automated. More specifically, as a sample is added to an inlet (which may include a filtration membrane or similar element), the fluid starts the sequential reagent delivery and washing cycles without any further intervention by the user. Accordingly, the only substantive steps to be performed by the end-user to execute a multiplexed assay are the addition of the sample, which may contain a buffer (e.g., sample diluted in a sample buffer) As previously discussed, in at least certain embodiment, after addition of the fluid sample, all channels of the microfluidic network may be filled with the fluid, which may preferably include or combined buffer and sample, or optionally a separate plug of sample and buffer, due to capillary action. Notably, the microfluidic network may include vents or similar openings (e.g., above the dried reagent pads) to ensure proper filling of the assay device and to ensure venting of air to prevent bubbles that may impede flow through the microfluidic network, except as otherwise provided for. Once the channels are filled, passive pump (e.g., a waste pad, nitrocellulose body of a test strip, etc.) may be coupled to the microfluidic network or otherwise made to contact the fluid within the microfluidic network, thereby pumping/drawing fluid through the microfluidic network to a testing or detection zone (e.g., of a test strip) configured include at least two test strips each configured to detect a distinct analyte. In at least certain implementations, the sample inlet is placed immediately upstream of the detection zone such that the sample is delivered to the detection zone first.

Multiplex assay devices according to this disclosure may include two or more dried reagents, which may be stored on pads within the microfluidic network or otherwise disposed within the microfluidic network, and preferably in adjacent microfluidic pathways configured to operate independently in parallel with one another to detect a distinct analyte. In certain embodiment, a first of the dried reagents may be an enzyme or nanozyme label while a second of the dried reagents may be a substrate. The microfluidic network is generally configured such that after introduction of a sample, the sample may be transported to the detection zone by capillary action. A portion of the fluid may follow, thereby washing away excess sample that may interfere with the remaining assay. Following introduction of the fluid, dried reagent stored within the microfluidic network may be rehydrated by the buffer fluid and generally permitted to flow toward the detection zone. In a two-reagent configuration, the pressure differential or other parameter impacting flow is such that rehydrated reagent from the first reagent pad (e.g., enzyme label) arrives at the detection zone before rehydrated reagent from the second reagent pad. As a result, target analyte captured in the detection zone may capture the rehydrated reagent form the first reagent pad. Following delivery of the first reagent, the second reagent (e.g., rehydrated substrate) may be delivered to its corresponding detection zone. The rehydrated substrate may optionally be preceded at the detection zone by a quantity of fluid, which washes away excess of the first reagent. When the rehydrated second reagent reaches the detection zone, the rehydrated second may react with the first reagent, producing a visible color change or similar effect. In certain embodiment, after flow is substantially complete in the device, the color change may generally be detectable with the naked eye for qualitative detection, imaged (e.g., using a smartphone camera) for quantitative information, or otherwise interpreted.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

As used herein, the term “comprise,” or variations thereof such as “comprises” or “comprising,” are to be read to indicate the inclusion of any recited integer (e.g., a feature, element, characteristic, property, method/process step or limitation) or group of integers (e.g., features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “microfluidic chip” means a device for manipulating nanoliter to microliter volumes of liquid. Such devices frequently contain features such as channels, chambers, and/or valves, and can be fabricated from a variety of different materials, including, but not limited to, glass and polydimethylsiloxane (PDMS).

A “channel,” as used herein, means a feature on or in an article (substrate) that at least partially directs the flow of a fluid. The channel can have any cross-sectional shape (circular, oval, triangular, irregular, square or rectangular, or the like) and can be covered or uncovered. In embodiments where it is completely covered, at least one portion of the channel can have a cross-section that is completely enclosed, or the entire channel may be completely enclosed along its entire length with the exception of its inlet(s) and outlet(s). A channel may also have an aspect ratio (length to average cross sectional dimension) of at least 2:1, more-typically at least 3:1, 5:1, or 10:1 or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to fluid flow of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases, the dimensions of the channel may be chosen such that fluid is able to freely flow through the article or substrate. The dimensions of the channel may also be chosen, for example, to allow a certain volumetric or linear flowrate of fluid in the channel. Of course, the number of channels and the shape of the channels can be varied by any method known to those of ordinary skill in the art. In some cases, more than one channel or capillary may be used.

For example, two or more channels may be used, where they are positioned inside each other, positioned adjacent to each other, positioned to intersect with each other, etc.

“Analyte” means any chemical compound, biomolecule, bacteria, virus or portions thereof susceptible to detection and/or quantification through the device or methods of the invention. “Sample” means a composition that may or may not include an analyte.

As used herein, the term “sample” or “fluid” includes any fluid to be tested by the devices or methods of the invention, and may preferably include biological or environmental fluid samples, such as wastewater sample, and preferably wastewater samples containing a biological or chemical analyte. A “biological sample” includes any bodily fluid or tissue. Biological samples or samples appropriate for use according to the methods provided herein include, without limitation, blood, serum, urine, saliva, tissues, cells, and organs, or portions thereof, as well as isolated cells derived from a subject, or other organism, such as a bacterium, plant, fungi or other cell. Additional embodiment can include bone marrow, such as bone marrow aspirates, as well as cell, tissues or fluid aspirates, including fine needles aspirates. A “subject” is any organism of interest, generally a mammalian subject, and preferably a human subject.

As used herein the singular forms “a”, “an”, and “the” include plural referents unless the context clearly dictates otherwise. Thus, for example, reference to “a compound” includes a plurality of such compounds, and reference to “the method” includes reference to one or more methods, method steps, and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or +a range of up to 20%, up to 15%, up to 10%, up to 5%, or up to 4%, 3%, 2%, or 1 % compared to the specifically recited value.

The invention now being generally described will be more readily understood by reference to the following examples, which are included merely for the purposes of illustration of certain embodiments of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1

### Multiplexed Assay Summary Results and Methodological Framework

The present inventors describe a capillary-driven device that passively delivers the reagent and washing steps associated with an ELISA to two, spatially separated test strips with visual results in under 15 min from a single sample addition step. The device, in one embodiment uses horseradish peroxidase (HRP) as a signal enzyme, and 3,3′,5,5′-tetramethylbenzydine (TMB) as the amplification substrate. The device, in one embodiment, is assembled from inexpensive double-sided adhesive and transparency film to create the microfluidic front end that leads to two nitrocellulose test strips. The channels of the device are oriented in such a way that, after sample addition, sample, signal antibody, wash steps, and substrate are delivered to two separate test strips simultaneously. The device is also designed so reagents for one target do not interact with reagents from the other side of the device. This separation prevents cross reactivity that may occur between capture and signal antibodies from the other assay.

As described below, multiplexed detection was shown in two motifs. The first demonstrates the device's ability to distinguish between two conditions. For this example of multiplexed detection, the device was used to distinguish between H1N1 and SARS-COV-2 infections from a single sample. These two are of special interest because many of their symptoms (fever, muscle soreness, cough, headaches, nausea, etc.) are not specific to either disease, making it difficult to distinguish between the two solely on the symptoms a patient presents. In this embodiment, the device was optimized to detect hemagglutinin from H1N1 on one test strip, and nucleocapsid protein (N-protein) from SARS-COV-2 on the other. Initial optimization for the H1N1 assay was performed on a single-channel device as described by Henry et al. Once conditions were chosen, dose response curves were generated for H1N1 hemagglutinin and SARS-CoV-2 nucleocapsid protein (N-protein) on the multiplex device with detection limits of 840 pg/mL and 133 pg/mL respectively. The amount of SARS-COV-2 N-protein found in blood and saliva from positive patients ranges from 1 to 1×10⁵ PFU/mL during the first two weeks after infection; our device is well within that range. Although it is difficult to determine hemagglutinin concentration in positive patients, other similar POC devices claim LODs of 0.23-29 ng/mL. To show the devices function in complex sample matrix, devices were run in nasal swabs spiked with varying concentrations of N-protein or HA. Results for our device can be read visually in under 15 min and can be quantified with a smartphone.

The second motif of multiplexed detection is simultaneous detection of two targets. To demonstrate this type of multiplexed detection, two examples are shown. First would be an example of a coinfection of SARS-COV-2 and H1N1. Although coinfections of the two diseases are rare, many cases have been reported, and the severity of patient outcomes is typically more severe and a higher risk of death. To demonstrate simultaneous detection devices were run with 5 and 100 ng/ml of both N-protein and HA simultaneously to show simultaneous detection of both viral proteins. To further demonstrate this concept, N-protein from SARS-COV-2 and C-reactive protein (CRP) were also detected simultaneously. Elevated levels of CRP have been found in patients infected with SARS-COV-2. Consequently, it has been suggested that elevated levels of CRP could be used as a predictor for the severity of a patient's condition. Therefore, simultaneously detecting a SARS-COV-2 infection and quantifying the amount of CRP in the same sample may be useful to the decision making of healthcare providers. For this example, samples containing N-protein at 50 ng/mL and varying amounts of CRP were run. There was no statistical difference in the signal from the N-protein test strip, and the CRP test strip signal increased according to the increase in CRP concentration. This work demonstrates the function and some potential applications of multiplexed, capillary driven immunoassays for multiple biomarkers detected at clinically relevant concentrations.

### Example 2

### Assay Optimization

As noted above, multiplexed analysis in medical diagnostics is widely accepted as a more thorough and complete method compared to single analyte detection. While analytical methods like PCR and ELISA exist for multiplexed detection of biomarkers, they remain time consuming and expensive. Lateral flow assays (LFAs) are an attractive option for point-of-care testing, and examples of multiplexed LFAs exist. However, these devices are limited by spatial resolution of test lines, large sample volumes, cross-reactivity, and poor sensitivity. As described by Henry et al., capillary-flow microfluidic ELISA platforms are a more sensitive alternative to LFAs. However, multiplexed detection on these types of devices has yet to be demonstrated. In the aftermath of the initial SARS-COV-2 pandemic, the need for rapid, sensitive point of care devices has become apparent. Moving forward, devices that are able to distinguish between diseases with similar presenting symptoms would be the ideal home diagnostic. As described below, the preset invention describes a multiplexed capillary-flow immunoassay device configured for the simultaneous detection of multiple biomarkers. From a single sample addition step, the reagents and washing steps required for two simultaneous ELISAs are delivered to spatially separated test strips. As further described herein, visual results of the device can be obtained in <15 min, and in optional embodiments, images of the assay results can be captured with a smartphone for processing by a computer executable program to generate additional quantitative data.

In one exemplary embodiment of the present invention, Applicants describe a spatially separated, capillary driven ELISA that can detect SARS-COV-2 nucleocapsid protein (N-protein) and influenza A H1N1 hemagglutinin simultaneously within 15 minutes from a single sample addition. The device uses horseradish peroxidase (HRP) as a signal enzyme, and 3,3′,5,5′-tetramethylbenzydine (TMB) as the amplification substrate. The device is assembled from inexpensive double-sided adhesive and transparency film to create the microfluidic front end that leads to two nitrocellulose test strips. The channels of the device are oriented in such a way that, after sample addition, sample, signal antibody, wash steps, and substrate are sequentially delivered to two separate test strips, which may or may not be simultaneously. The multiplexed assay device of the invention is also designed so that reagents for one target do not interact with reagents from on the other side of the device. This separation prevents cross reactivity that may occur between capture and signal antibodies from the other assay.

As described below, initial optimization for the H1N1 assay was performed on a single-microfluidic channel device similar to that described by Henry et al. Once conditions were chosen, dose response curves were generated for H1N1 hemagglutinin and SARS-COV-2 nucleocapsid protein (N-protein or NP) were performed on the multiplexed assay device of the invention with detection limits of 840 pg/mL and 133 pg/mL respectively. To show the devices function with a complex sample matrix, devices were run in nasal swabs spiked with varying concentrations of N-protein or HA.

Finally, exemplary multiplex assay devices were run with 5 and 100 ng/ml of both N-protein and HA simultaneously to further demonstrate simultaneous detection of at least two target analytes. In another similar example, N-protein from inactivated virus and CRP were also detected simultaneously. In both cases, signal was present on both test strips, demonstrating this device's use for the detection multiple analytes simultaneously.

In the preferred embodiment shown in the figures, the multiplex device of the invention consists of two, separate immunoassays. To optimize the HA assay, the single channel CaDI device described by Henry et al., was used, and the location of conjugate release pads and NC membrane are chosen in FIG. 2A. Manufacturing the single channel devices in bulk was slightly easier, giving us a quick way to determine the optimal 2°Ab-HRP concentrations and TMB volumes to use. First, the volume of TMB dried on the TMB pad was held constant at 30 μL while the concentration of 2° Ab-HRP dried was varied. The optimal concentration of 2° Ab-HRP was based on the results that gave the greatest signal for 50 ng/ml HA in running buffer and no signal with 0 ng/ml HA. The lowest concentration of 2° Ab-HRP where those criteria were met was 40 μg/mL. Despite having excess 2°Ab-HRP, more than 40 μg/mL did not improve signal and had a slightly higher signal for the blank samples (FIG. 2B). Next, 2°Ab-HRP was held constant at 40 μg/mL while the volume of TMB substrate dried was varied. Devices were again run at 0 and 50 ng/ml HA. Although all TMB volumes above 15 μL performed similarly for signal intensity, 22.5 μL was chosen to ensure an excess of TMB is present in the device (FIG. 2B). Along with benefiting the multiplex device, this HA assay development is the first example of an automated, capillary driven immunoassay for HA.

### Example 3

### Multiplex Device Function

The optimized reagent pad conditions for an N-protein assay and HA assay were incorporated into the multiplexed microfluidic device. A NC test strip for each assay was inserted on its corresponding side, and the device was pressed down on an adhesive backing with a waste pad connected to the membrane. To operate the device, 160 μL of sample is added to the sample inlet. No other end user steps are required after sample addition. When sample is added, the solution fills the hollow channels in the device. Sample rehydrates 2° Ab-HRP and TMB from the conjugate release pads and flows through each NC test strip. If either target is present, it will bind to the capture antibody striped on its NC membrane. After this initial filling step, the buffer in the channels begins to drain into the waste pad through capillary forces of the waste pad and the NC membranes. As the channels drain, an air bubble forms in the center channel above the sample inlet. Because of this air gap, 2° Ab-HRP from one side of the device cannot cross over to the other. 2° Ab-HRP is the next reagent delivered to the NC, followed by a gap of sample buffer that washes any excess from the test lines. Lastly, TMB is delivered to each test line. The assay operation is shown in FIG. 3A and B with blue and yellow food dye dried on the conjugate release pads to help visualize flow. Mismatched colors were chosen to show that there was no crossover from one side of the device to the other when reagents are delivered to the test strips. If target is present at one or both test lines, 2° Ab-HRP will also be present. TMB can react with HRP and H₂O₂ in the running buffer to form a solid blue test line. Control lines consisting of anti-mouse and anti-rabbit antibodies for NP and HA assay respectively can be present.

### Example 4

### Dose Response Curves and Simultaneously Detection

Separate dose response curves were gathered for both sides of the device. One target was diluted in a running buffer containing hydrogen peroxide for the HRP reaction. The running buffer also contains NaCl for protein stability, and surfactants to encourage release of reagents from the conjugate release pads, prevent non-specific binding on the test lines and to encourage uniform flow through the nitrocellulose, although some nonuniformity can be seen in the test lines. Varying concentrations of N-protein were run in triplicate with no HA present, and the resulting signal was fit to 4 parameter logistic regression (4 PL). The LOD for N-protein was determined to be three standard deviations above the signal of the blank sample, meaning the LOD was 133 pg/mL. Ideally, regardless of the increase in concentration of N-protein, the signal in the HA assay NC membrane should never be present if only N-protein is present in the sample. If that is the case, a linear fit to the HA data should have a slope of 0. In this case, the slope of a linear fit to the HA signal was 0.00823 (FIG. 4A). Additionally, an analysis of variance on the HA data shows the points are indistinguishable from each other with 95% confidence. The same procedures were done holding the N-protein concentration at 0 and varying the concentration of HA. For that data, the LOD of HA is 840 pg/mL, the slope of a linear fit for the N-protein side of the device is 0.00118, and the analysis of variance on the N-protein data showed the points were indistinguishable with 95% confidence (FIG. 4B).

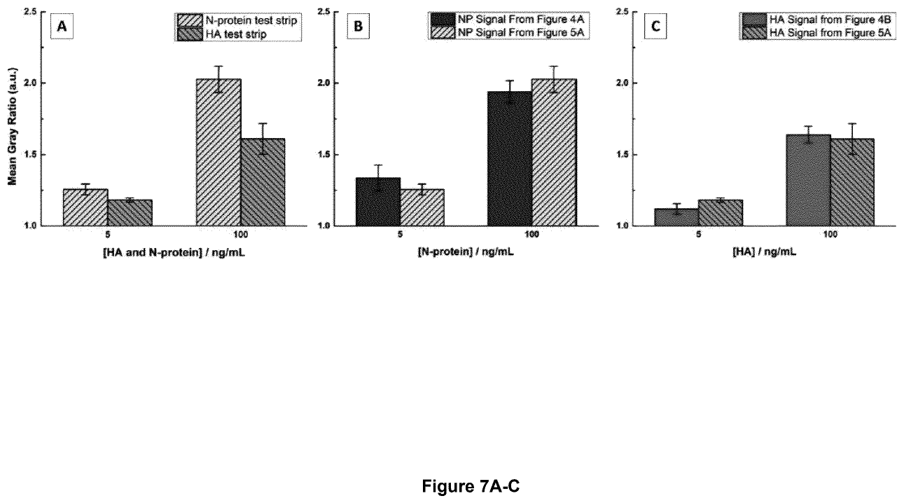

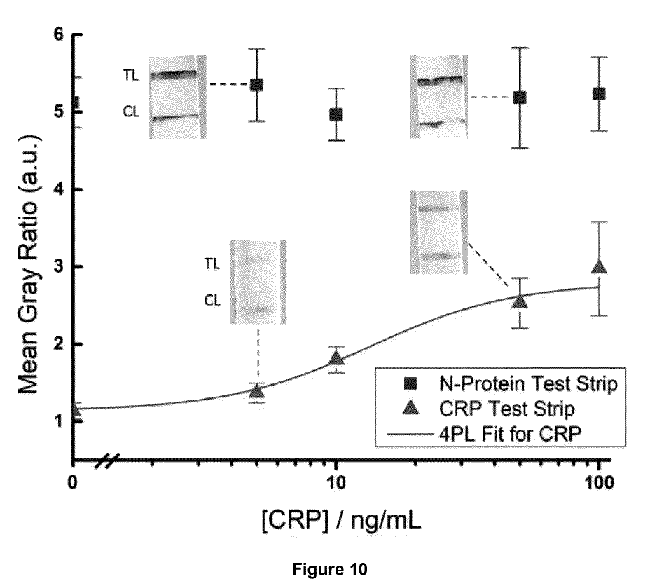

Recent reports have shown that co-infection of influenza and SARS-COV-2 occurs in a significant number of cases. To demonstrate the effectiveness of this multiplex device to detect two analytes at the same time, HA and N-protein were spiked into running buffer. These samples containing both targets were run in triplicate, and the images for both sides of the devices were processed as described above (FIG. 7A). At both concentrations, signal was present on both test strips after the samples had run. The resulting signal from FIG. 7A was also compared to the signal that was obtained from the dose response curve data in FIG. 4 where only one target was present in sample. With overlapping error bars and an analysis of variance, there is no significant difference in the signal between the data in FIG. 4 and FIG. 7A (FIGS. 7B and 7C). This means that the presence of one target in sample does not hinder the other side's ability to detect its intended target. Continuing to expand on multiplex applications, simultaneous detection of SARS-CoV-2 and CRP was investigated next. Elevated levels of CRP in patients infected with SARS-CoV-2 has been correlated with worse patient outcomes. Therefore, simultaneous diagnosis of a SARSCOV-2 infection and quantification of CRP may help in prognosis and treatment. Devices were assembled with SARS-COV-2 reagents and test strip on one side, and with CRP reagents and test strip on the other. Extraction buffer was spiked with varying concentrations of CRP as well as 50 ng/mL N-protein before running in the multiplex device. 50 ng/mL N-protein represents a concentration at the high end of the dose response in FIG. 4A, and CRP concentrations were varied from 0 -100 ng/ml (FIG. 10). The presence of N-protein did not cause false positives on the CRP test strip, and the CRP test strip signal increased as CRP concentration increased. Likewise, increasing the CRP concentration did not affect the signal present on the N-protein test strip, with an analysis of variance showing no statistical difference between the signal at any CRP concentration with >95% confidence. In this instance, the LEDs for the lightbox used to image were replaced before imaging, leading to a lighter background and a higher signal ratio on the N-protein side of the device compared to FIG. 4. Ideally with this type of assay, health care providers would be able to determine if someone has a SARS-COV-2 infection and how severe their symptoms may be based on quantification of CRP.

To demonstrate the effectiveness of this multiplexed assay device to detect two analytes at the same time, one relatively low and one relatively high concentration of both HA and N-protein was spiked into running buffer. These samples containing both targets were run in triplicate, and the images for both sides of the devices were processed as described above (FIG. 7). At both concentrations, signal was present on both test strips after the devices had run. The presence of one target did not hinder the other side's ability to detect its intended target. Continuing the expansion of multiplex applications, simultaneous detection of SARS-COV-2 and CRP was investigated. Elevated levels of CRP in patients infected with SARS-COV-2 has been correlated with worse patient outcomes. Therefore, simultaneous diagnosis of a SARS-COV-2 infection and quantification of CRP would greatly help in prognosis and treatment. Devices were assembled with SARS-COV-2 reagents and test strip on one side, and with CRP reagents and test strip on the other. Extraction buffer was spiked with 1100 PFU/mL of inactivated SARS-COV-2 and 100 ng/mL CRP before running in the multiplex assay device of the invention. (FIG. 6) As a control, one device was run with no sample present.

The present inventors demonstrated the first example of a multiplexed capillary flow microfluidic ELISA in a POC-style device. Using hollow microfluidic channels, spatially separated NC test strips, and conjugate release pads device operates in a single sample addition step and outputs visually detectable signal in under 15 min. The device can detect HA from H1N1 in with an LOD of 840 pg/mL and N-protein from SARS-COV-2 with an LOD of 133 pg/mL when spiked in running buffer. The device was initially designed with differentiation between SARS-CoV-2 and H1N1 infections in mind, the device can also be used for simultaneous detection of two targets. To demonstrate this concept, N-protein and HA were detected simultaneously, as well as simultaneous detection of N-protein and CRP. This work demonstrates two important applications of multiplexed diagnostics: 1) distinguishing between conditions that may share similar symptoms, and 2) simultaneous detection of two targets for more thorough analysis and future patient care. In all examples shown in this work, signal is visible by naked eye in under 15 min, and quantitative data can be determined using smartphone images.

### Example 5

### Materials and Methods

Nitrocellulose test strip preparation: Nitrocellulose (NC) membranes (Vivid 90 LFNC, Pall, NY, USA) were striped with test and control lines for each assay. For the N-protein test strips, 0.88 mg/mL of anti-N-protein capture antibody (40143-MM08, Sino Biological, Beijing China) and goat anti-mouse control line antibody (AB6708, Abcam, Cambridge UK) were striped using a BioSpot reagent printer (BioFluidix, Breisgau, Germany). For the HA test strips, 0.88 mg/mL of anti-HA capture antibody (11055-RM10, Sino Biological) and goat anti-rabbit control line antibody (R1131, Millipore Sigma, Burlington MA, USA) were striped. For the CRP test strips, 0.88 mg/mL anti-CRP capture antibody (PAB 7943, Abnova, Taipei, Taiwan) and a CRP (30-AC05S, Fitzgerald, Acton MA, USA) control line was striped. All test line and control line solutions also contained 4% glycerol and 4 mM trehalose for improved drying and longevity. All NC membranes were dried overnight in a desiccator before blocking with Stabilguard™ (SG01-1000, Surmodics Inc.) to prevent nonspecific adsorption to the membrane during the assay. The NC membranes were then cut into 15×3 mm2 individual test strips using a CO2 laser cutter (Epilog Zing, Golden, CO, USA). NC membranes were stored at 4° C. with desiccant until used.

Reagent pad preparation: Glass fiber pads (GFDX203000, MilliporeSigma Burlington, MA, USA) were used for both TMB and secondary antibody-HRP (2° Ab-HRP) pads, but the pretreatment/blocking conditions for each were slightly different. For TMB pad, glass fiber sheets were completely submerged in a blocking solution containing 10 mM PBS (Thermo Scientific, Rockford, IL, USA), 3% sucrose, 0.5% Tween-20, and 0.1% thimerosal (MilliporeSigma) for 15 min. The sheets were removed from the solution and dried overnight at 37° C. before use. The dried, blocked membranes were then cut into 3×5 mm2 reagent pads with a razor blade. TMB (TMBM-0100-01, Surmodics Inc., Eden Prairie, MN, USA) was pipetted onto the pads in 7.5 μL aliquots, drying for 7-8 min between additions. After the final TMB addition, the pads were dried for 2 h before assembling in the microfluidic device. For 2° Ab-HRP pads, blank glass fiber membranes were cut into 3×5 mm2 reagent pads before any pretreatment. Aged casein was prepared following the procedures outlined in work from Grant et. al. The aged casein was diluted to 0.5% in 10 mM PBS, and 15 μL was added to each 2° Ab-HRP pad in two, 7.5 μL aliquots. The pads were dried for 7-8 min at 37° C. between additions, and for an additional 30 minutes after the second. 2° Ab-HRP for the N-protein assay (40143-MM05-H, Sino Biological) and the HA assay (11055-RP07, Sino Biological) were diluted in a drying buffer designed for long-term stability. 5 μL of each diluted 2° Ab-HRP was added to separate treated pads and dried for 30 min at 37° C. before adding to the device. For the CRP assay, a conjugation kit (ab 102890, Abcam, Cambridge UK) was used to conjugate HRP to the detection antibody (MAB0421, Abnova). The manufacturer's protocol was followed for the conjugation kit, and the conjugated antibody was diluted to 20 μg/mL in drying buffer. 5 μL of the diluted Ab-HPR solution was added to treated glass fiber pads and dried for 30 min at 37° C.

Device assembly: Patterns for the device channels were designed in the CAD program, OnShape, and cut into alternating layers of transparency film (3M™ 9984, Saint Paul, MN, USA) and double-sided adhesive (3M™ 467MP, Saint Paul, MN, USA) with a CO₂ laser cutter (FIGS. 1A and 1B). The first four layers of the device were assembled before the conjugate release pads were added. The final layer of transparency film was added last, completing the hollow channels and securing the conjugate release pads in place. The devices were cut at the bottom to open the channels for the insertion of the NC test strips. Conjugate release pads and NC test strip for the HA assay were assembled into the left half of the device, and N-protein pads and test strip were assembled into the example shown in FIG. 1C. The entire device was adhered to an adhesive backing, and a waste pad (CFSP223000, Millipore Sigma, Burlington MA, USA) was pressed down, overlapping with the end of the NC membrane.

Assay optimization: The N-protein assay has been previously studied extensively. Therefore, the concentrations of 2° Ab-HRP and volume of TMB substrate used in the N-protein assay were the same as from previous work. The running buffer that sample is diluted in is made using 1.5× stable peroxide buffer (Thermo Fisher, 34062) at pH 6.5 with 150 mM NaCl, 0.1% Igepal CA630, and 0.1% Tween-20. The influenza A H1N1 assay has never been performed on this type of capillary driven immunoassay device (CaDI). To optimize the conditions for the HA side of the multiplex device, the HA assay was performed on the single channel CaDI device outlined by Henry et al., described above. First, TMB volume was held constant at 30 μL while the 2° Ab-HRP concentration was adjusted between 5 and 60 μg/mL. Devices were run in triplicate with running buffer containing either 0 or 50 ng/ml HA (11085-V08B, SinoBiological). 100 μL of sample was added to the sample inlet, and when the devices had completed, the NC was removed and imaged with a smartphone (Motorola 1) under a lightbox containing 16 LEDs. Based on the analyzed results, a concentration of 40 μg/mL for anti-HA 2° Ab-HRP was chosen. For TMB optimization, the anti-HA 2° Ab-HRP was held constant at 40 μg/mL while the TMB volume was varied from 7.5 -37.5 L. Devices were run again in triplicate with 0 or 50 ng/ml HA, and 22.5 μL of TMB was chosen moving forward. For the CRP assay, a concentration of 20 μg/mL anti-CRP 2° Ab-HRP and a volume of 22.5 μL of TMB were chosen. These conditions worked considerably well for the CRP assay with litter further optimization.

Multiplex assay operation: After HA assay conditions were optimized in the single-channel CaDI, dose response curves for both assays were collected in the multiplex device. First, a range of HA concentrations were diluted in running buffer, and 160 μL of each concentration was added to the sample inlet of the multiplex device. Sample completely fills the channels and delivers target to both NC test strips. Afterwards, reagents for each side of the device are delivered passively to the NC membrane from the device via capillary action driven by the absorbent waste pad. The assay is complete when the buffer above the TMB pads is drained from the channels through the NC membrane, to the waste pad. After the assay was complete (<15 min), NC membranes were removed, allowed to dry for 5 min at RT before imaging with a Motorola 1 smartphone under the light box. The same procedures were performed with varying concentrations of N-protein spiked into running buffer. Devices were also run with 5 and 100 ng/ml of both targets simultaneously spiked into running buffer as a demonstration of simultaneous detection of two targets present in the same sample. As another example of simultaneous detection, 50 ng/ml of SARS-COV-2 N-protein was added to running buffer already containing varying concentrations of CRP. One side of the multiplex device was prepared with the N-protein conjugate release pads and NC test strip while the other contained reagents and NC for the CRP assay described above.

Spiked Nasal Swab Samples: Anterior nares swab was performed under IRB approved conditions. A foam swab (FoamTec Medical, MP1301AST) was swirled in each nostril 3-5×. To each swab, 50 μL of extraction buffer spiked with N-protein, HA, or both was added to the swab. The swabs were submerged in 300 μL of running buffer for 15 s and then pressed through a 0.2 um syringe filter (Fisher, 13-1001-14). 160 μL of the filtered sample was then added to the multiplexed device. When devices had finished, the NC membranes were removed and allowed to dry at RT for 5 min. NC membranes were imaged under an LED lightbox with a smartphone (Motorola 1).

Image/Data Analysis: NC membranes were allowed to dry for 5 min at RT before being imaged under a light box. Images were uploaded to the free NIH software, ImageJ. Images were converted to 8-bit gray scale and inverted to provide a positive correlation with increasing target concentration. A rectangular area of interest was drawn around the area of the test line (or the blank area where the test line would be in the case of blank samples). A mean gray value measurement was taken of the test line area, and another measurement was taken in the area between the test line and control line. The ratio of these values is called the “mean gray ratio” and was used for quantification purposes.

### REFERENCES

(1) Rasmussen, J.; Langerman, H. Alzheimer's Disease—Why We Need Early Diagnosis. Degenerative neurological and neuromuscular disease 2019, 9, 123-130.
(2) Kashyap, D.; Kaur, H. Cell-free miRNAs as non-invasive biomarkers in breast cancer: Significance in early diagnosis and metastasis prediction. Life Sciences 2020, 246, 117417.
(3) Bohunicky, B.; Mousa, S. A. Biosensors: the new wave in cancer diagnosis. Nanotechnology, Science and Applications 2011, 4, 1-10.
(4) Clinton, W. J. Turning the Tide on the AIDS Pandemic. New England Journal of Medicine 2003, 348 (18), 1800-1802.
(5) Gayle, H. D. Curbing the Global AIDS Epidemic. New England Journal of Medicine 2003,348 (18), 1802-1805.
(6) Nitsche, A.; Schweiger, B.; Ellerbrok, H.; Niedrig, M.; Pauli, G. SARS coronavirus detection. Emerg Infect Dis 2004, 10 (7), 1300-1303.
(7) Al Hajjar, S.; McIntosh, K. The first influenza pandemic of the 21st century. Annals of Saudi medicine 2010, 30 (1), 1-10.
(8) Otoo, J. A.; Schlappi, T. S. REASSURED Multiplex Diagnostics: A Critical Review and Forecast. Biosensors (Basel) 2022, 12 (2).
(9) Petti, C. A.; Polage, C. R.; Quinn, T. C.; Ronald, A. R.; Sande, M. A. Laboratory Medicine in Africa: A Barrier to Effective Health Care. Clinical Infectious Diseases 2006, 42 (3), 377-382.
(10) Herd, G. C. E., Musaad, S. M. A. Point-of-Care Testing in Rural and Remote Settings to Improve Access and Improve Outcomes: A Snapshot of the New Zealand Experience. Archives of Pathology & Laboratory Medicine 2020, 145 (3), 327-335.
(11) Noviana, E.; Ozer, T.; Carrell, C. S.; Link, J. S.; McMahon, C.; Jang, I.; Henry, C. S. Microfluidic Paper-Based Analytical Devices: From Design to Applications. Chemical Reviews 2021, 121 (19), 11835-11885.
(12) Omidfar, K.; Riahi, F.; Kashanian, S. Lateral Flow Assay: A Summary of Recent Progress for Improving Assay Performance. Biosensors (Basel) 2023, 13 (9).
(13) Sena-Torralba, A.; Alvarez-Diduk, R.; Parolo, C.; Piper, A.; Merkoçi, A. Toward Next Generation Lateral Flow Assays: Integration of Nanomaterials. Chemical Reviews 2022, 122 (18), 14881-14910.
(14) Jara, M. D. L.; Alvarez, L. A. C., Guimarães, M. C. C.; Antunes, P. W. P.; de Oliveira, J. P. Lateral flow assay applied to pesticides detection: recent trends and progress. Environmental science and pollution research international 2022, 29 (31), 46487-46508.
(15) Ngom, B.; Guo, Y.; Wang, X.; Bi, D. Development and application of lateral flow test strip technology for detection of infectious agents and chemical contaminants: a review. Anal Bioanal Chem 2010, 397 (3), 1113-1135.
(16) Chen, Y.; Chen, Q.; Han, M.; Zhou, J.; Gong, L.; Niu, Y.; Zhang, Y.; He, L.; Zhang, L. Development and optimization of a multiplex lateral flow immunoassay for the simultaneous determination of three mycotoxins in corn, rice and peanut. Food Chemistry 2016, 213, 478-484.
(17) Tominaga, T. Rapid detection of coliform bacteria using a lateral flow test strip assay. Journal of Microbiological Methods 2019, 160, 29-35.
(18) Zhao, Y.; Wang, H.; Zhang, P.; Sun, C.; Wang, X.; Wang, X.; Yang, R.; Wang, C., Zhou, L. Rapid multiplex detection of 10 foodborne pathogens with an up-converting phosphor technology-based 10-channel lateral flow assay. Scientific Reports 2016, 6 (1), 21342
(19) Yin, M.; Nie, Y.; Liu, H.; Liu, L.; Tang, L.; Dong, Y.; Hu, C.; Wang, H. Development of a europium nanoparticles lateral flow immunoassay for NGAL detection in urine and diagnosis of acute kidney injury. BMC Nephrology 2022, 23 (1), 30.
(20) Koczula, K. M.; Gallotta, A. Lateral flow assays. Essays Biochem 2016, 60 (1), 111-120.
(21) Peng, J.; Wang, Y.; Liu, L.; Kuang, H.; Li, A.; Xu, C. Multiplex lateral flow immunoassay for five antibiotics detection based on gold nanoparticle aggregations. RSC Advances 2016, 6 (10), 7798-7805, 10.1039/C5RA22583C.
(22) Wang, Q.; Liu, Y.; Wang, M.; Chen, Y.; Jiang, W. A multiplex immunochromatographic test using gold nanoparticles for the rapid and simultaneous detection of four nitrofuran metabolites in fish samples. Analytical and Bioanalytical Chemistry 2018, 410 (1), 223-233.
(23) Parolo, C.; Sena-Torralba, A.; Bergua, J. F.; Calucho, E.; Fuentes-Chust, C.; Hu, L.; Rivas, L.; Álvarez-Diduk, R.; Nguyen, E. P.; Cinti, S.; Quesada-González, D.; Merkoçi, A. Tutorial: design and fabrication of nanoparticle-based lateral-flow immunoassays. Nature Protocols 2020, 15 (12), 3788-3816.
(24) Ragavendar, M. S.; Anmol, C. M. A mathematical model to predict the optimal test line location and sample volume for lateral flow immunoassays. In 2012 Annual International Conference of the IEEE Engineering in Medicine and Biology Society, 28 Aug.-1 Sept. 2012, 2012; pp 2408-2411.
(25) Washburn, E. W. The Dynamics of Capillary Flow. Physical Review 1921, 17 (3), 273-283.
(26) Gomez-Martinez, J.; Silvy, M.; Chiaroni, J.; Fournier-Wirth, C.; Roubinet, F.; Bailly, P.; Brès, J.-C. Multiplex Lateral Flow Assay for Rapid Visual Blood Group Genotyping. Analytical Chemistry 2018, 90 (12), 7502-7509.
(27) Taranova, N. A.; Byzova, N. A.; Zaiko, V. V.; Starovoitova, T. A.; Vengerov, Y. Y.; Zherdev, A. V.; Dzantiev, B. B. Integration of lateral flow and microarray technologies for multiplex immunoassay: application to the determination of drugs of abuse. Microchimica Acta 2013, 180 (11), 1165-1172.
(28) Dincer, C.; Bruch, R.; Kling, A.; Dittrich, P. S.; Urban, G. A. Multiplexed Point-of-Care Testing-xPOCT. Trends Biotechnol 2017, 35 (8), 728-742.
(29) Goldmann, F.; Bauer, N.; Moritz, A. Evaluation of the IDEXX ProCyte Dx analyzer for dogs and cats compared to the Siemens ADVIA 2120 and manual differential. Comparative Clinical Pathology 2014, 23 (2), 283-296.
(30) Ernst, R.; Ogeer, J.; McCrann, D.; Cross, J.; Strong-Townsend, M.; Friis, H.; Coyne, M.; Clements, C.; Drake, C.; Murphy, R. Comparative performance of IDEXX SDMA Test and the DLD SDMA ELISA for the measurement of SDMA in canine and feline serum. PLOS ONE 2018, 13 (10), e0205030.
(31) Hong, W.; Huang, L.; Wang, H.; Qu, J.; Guo, Z.; Xie, C., Zhu, Z.; Zhang, Y.; Du, Z.; Yan, Y.; Zheng, Y.; Huang, H.; Yang, R.; Zhou, L. Development of an up-converting phosphor technology-based 10-channel lateral flow assay for profiling antibodies against Yersinia pestis. Journal of Microbiological Methods 2010, 83 (2), 133-140.
(32) Han, D. K.; Oh, J.; Lee, J.; Cho, Y. G.; Park, J. S.; Choi, J. S.; Kim, D. S.; Kwon, J. Paper based multiplex analytical device for simultaneous detection of Clostridioides difficile toxins and glutamate dehydrogenase. Biosensors and Bioelectronics 2021, 176, 112894.
(33) Cheng, C.-M.; Martinez, A. W.; Gong, J.; Mace, C. R.; Phillips, S. T.; Carrilho, E., Mirica, K. A.; Whitesides, G. M. Paper-Based ELISA. Angewandte Chemie International Edition 2010, 49 (28), 4771-4774.
(34) Fabiani, L.; Mazzaracchio, V.; Moscone, D.; Fillo, S.; De Santis, R.; Monte, A.; Amatore, D.; Lista, F.; Arduini, F. Paper-based immunoassay based on 96-well wax-printed paper plate combined with magnetic beads and colorimetric smartphone-assisted measure for reliable detection of SARS-COV-2 in saliva. Biosensors and Bioelectronics 2022, 200, 113909.
(35) Verma, M. S.; Tsaloglou, M.-N.; Sisley, T.; Christodouleas, D.; Chen, A.; Milette, J.; Whitesides, G. M. Sliding-strip microfluidic device enables ELISA on paper. Biosensors and Bioelectronics 2018, 99, 77-84.
(36) Carrell, C. S.; Wydallis, R. M.; Bontha, M.; Boehle, K. E.; Beveridge, J. R.; Geiss, B. J.; Henry, C. S. Rotary manifold for automating a paper-based Salmonella immunoassay. RSC Advances 2019, 9 (50), 29078-29086, 10.1039/C9RA07106G.
(37) Mu, X.; Zhang, L.; Chang, S.; Cui, W.; Zheng, Z. Multiplex Microfluidic Paper-based Immunoassay for the Diagnosis of Hepatitis C Virus Infection. Analytical Chemistry 2014, 86 (11), 5338-5344.
(38) Samper, I. C.; Sánchez-Cano, A.; Khamcharoen, W.; Jang, I.; Siangproh, W.; Baldrich, E.; Geiss, B. J.; Dandy, D. S.; Henry, C. S. Electrochemical Capillary-Flow Immunoassay for Detecting Anti-SARS-COV-2 Nucleocapsid Protein Antibodies at the Point of Care. ACS Sensors 2021, 6 (11), 4067-4075.
(39) Clark, K. M.; Schenkel, M. S.; Pittman, T. W.; Samper, I. C.; Anderson, L. B. R.; Khamcharoen, W.; Elmegerhi, S.; Perera, R.; Siangproh, W.; Kennan, A. J.; Geiss, B. J.; Dandy, D. S.; Henry, C. S. Electrochemical Capillary Driven Immunoassay for Detection of SARS-COV-2. ACS Measurement Science Au 2022.
(40) Panraksa, Y.; Jang, I.; Carrell, C. S.; Amin, A. G.; Chailapakul, O.; Chatterjee, D.; Henry, C. S. Simple manipulation of enzyme-linked immunosorbent assay (ELISA) using an automated microfluidic interface. Analytical Methods 2022, 10.1039/D2AY00326K.
(41) Similarities and Differences between Flu and COVID-19. 2022. cdc.gov/flu/symptoms/flu-vs-covid 19.htm.
(42) Link, J. S.; Carrell, C. S.; Jang, I.; Barstis, E. J. O.; Call, Z. D.; Bellows, R. A.; O'Donnell-Sloan, J. J.; Terry, J. S.; Anderson, L. B. R.; Panraksa, Y.; Geiss, B. J.; Dandy, D. S.; Henry, C. S. Capillary flow-driven immunoassay platform for COVID-19 antigen diagnostics. Analytica Chimica Acta 2023, 1277, 341634.
(43) Shan, D.; Johnson, J. M.; Fernandes, S. C.; Suib, H.; Hwang, S.; Wuelfing, D.; Mendes, M.; Holdridge, M.; Burke, E. M.; Beauregard, K.; Zhang, Y.; Cleary, M.; Xu, S.; Yao, X.; Patel, P. P.; Plavina, T.; Wilson, D. H.; Chang, L.; Kaiser, K. M.; Nattermann, J.; Schmidt, S. V.; Latz, E.; Hrusovsky, K.; Mattoon, D.; Ball, A. J. N-protein presents early in blood, dried blood and saliva during asymptomatic and symptomatic SARSCOV-2 infection. Nature Communications 2021, 12 (1), 1931.
(44) Anderson, C. E.; Holstein, C. A.; Strauch, E.-M.; Bennett, S.; Chevalier, A.; Nelson, J.; Fu, E.; Baker, D.; Yager, P. Rapid Diagnostic Assay for Intact Influenza Virus Using a High Affinity Hemagglutinin Binding Protein. Analytical Chemistry 2017, 89 (12), 6608-6615.
(45) Chávez Ramos, K.; Nishiyama, K.; Maeki, M.; Ishida, A.; Tani, H.; Kasama, T.; Baba, Y.; Tokeshi, M. Rapid, Sensitive, and Selective Detection of H5 Hemagglutinin from Avian Influenza Virus Using an Immunowall Device. ACS Omega 2019, 4 (15), 16683-16688.
(46) Wang, Z.; Zhao, Q.; Huang, M.; Duan, Y.; Li, F.; Wang, T. Dual Detection of Hemagglutinin Proteins of H5N1 and H1N1 Influenza Viruses Based on FRET Combined With DNase I. Front Microbiol 2022, 13, Original Research.
(47) Covin, S.; Rutherford, G. W. Coinfection, Severe Acute Respiratory Syndrome Coronavirus 2 (SARSCOV-2), and Influenza: An Evolving Puzzle. Clinical Infectious Diseases 2020, 72 (12), e993-e994.
(48) Cuadrado-Payán, E.; Montagud-Marrahi, E.; Torres-Elorza, M.; Bodro, M.; Blasco, M.; Poch, E.; Soriano, A.; Pi\ddot{n}eiro, G. J. SARS-COV-2 and influenza virus co-infection. The Lancet 2020, 395 (10236), e84.
(49) Kim, E.-H.; Nguyen, T.-Q.; Casel, M. A. B.; Rollon, R.; Kim, S.-M.; Kim, Y.-I.; Yu, K.-M.; Jang, S.-G.; Yang, J.; Poo, H.; Jung, J. U.; Choi, Y. K. Coinfection with SARS-COV-2 and Influenza A Virus Increases Disease Severity and Impairs Neutralizing Antibody and CD4<sup>+</sup> T Cell Responses. Journal of Virology 2022, 96 (6), e01873-01821.
(50) Stowe, J.; Tessier, E.; Zhao, H.; Guy, R.; Muller-Pebody, B.; Zambon, M.; Andrews, N.; Ramsay, M.; Lopez Bernal, J. Interactions between SARS-COV-2 and influenza, and the impact of coinfection on disease severity: a test-negative design. International journal of epidemiology 2021, 50 (4), 1124-1133.
(51) Chen, N.; Zhou, M.; Dong, X.; Qu, J.; Gong, F.; Han, Y.; Qiu, Y.; Wang, J.; Liu, Y.; Wei, Y.; Xia, J. a.; Yu, T.; Zhang, X.; Zhang, L. Epidemiological and clinical characteristics of 99 cases of 2019 novel coronavirus pneumonia in Wuhan, China: a descriptive study. The Lancet 2020, 395 (10223), 507-513.
(52) Gao, Y.; Li, T.; Han, M.; Li, X.; Wu, D.; Xu, Y.; Zhu, Y.; Liu, Y.; Wang, X.; Wang, L. Diagnostic utility of clinical laboratory data determinations for patients with the severe COVID-19.Journal of Medical Virology 2020, 92 (7), 791-796.
(53) Ali, N. Elevated level of C-reactive protein may be an early marker to predict risk for severity of COVID-19. J Med Virol 2020, 92 (11), 2409-2411.
(54) Grant, B. D.; Anderson, C. E.; Williford, J. R.; Alonzo, L. F.; Glukhova, V. A.; Boyle, D. S.; Weigl, B. H.; Nichols, K. P. SARS-COV-2 Coronavirus Nucleocapsid Antigen-Detecting Half-Strip Lateral Flow Assay Toward the Development of Point of Care Tests Using Commercially Available Reagents. Analytical chemistry 2020, 92 (16), 11305-11309.
(55) Ramachandran, S.; Fu, E.; Lutz, B.; Yager, P. Long-term dry storage of an enzyme-based reagent system for ELISA in point-of-care devices. Analyst 2014, 139 (6), 1456-1462, 10.1039/C3AN02296J.
(56) Link, J. S.; Carrell, C. S.; Jang, I.; Barstis, E. J. O.; Call, Z. D.; Bellows, R. A.; O'Donnell-Sloan, J. J.; Terry, J. S.; Anderson, L. B. R.; Panraksa, Y.; Geiss, B. J.; Dandy, D. S.; Henry, C. S. Capillary flow-driven immunoassay platform for COVID-19 antigen diagnostics. Anal Chim Acta 2023, 1277, 341634.
(57) Cong, B.; Deng, S.; Wang, X.; Li, Y. The role of respiratory co-infection with influenza or respiratory syncytial virus in the clinical severity of COVID-19 patients: A systematic review and meta-analysis. Journal of Global Health 2022, 12.

1. A multiplex assay device comprising:
a microfluidic assembly including:
a sample inlet;
microfluidic network forming a plurality of microfluidic pathways each extending to a detection area, wherein each of said plurality of microfluidic pathways is in communication with said sample inlet, and further configured to detect a unique target analyte;
a first dried reagent disposed along each of said microfluidic pathways; and
a second dried reagent disposed each of said microfluidic pathways;
wherein, when a fluid is provided to the fluid inlet, the fluid fills the microfluidic network and directs a portion of the fluid through each of the microfluidic pathways to the detection area, and wherein the fluid rehydrates the first dried reagent disposed along each of said microfluidic pathways to produce a first rehydrated reagent, and rehydrates the second dried reagent disposed along each of said microfluidic pathways to produce a second rehydrated reagent, wherein the first rehydrated reagent and the second rehydrated reagent are sequentially delivered through their respective microfluidic pathways to the detection area by capillary-driven flow, and
wherein, when the fluid flows through the microfluidic network, an air bubble is formed within the network thereby restricting communication between the microfluidic pathways.

2. The device of claim 1, wherein said plurality of microfluidic pathways comprises two microfluidic pathways each configured to detect a unique target analyte.

3. The device of claim 2, wherein said first and second dried reagents are each contained on a conjugate release pad positioned within each of the microfluidic pathway.

4. The device of claim 2, wherein said two microfluidic pathways comprise two outer channels and two inner channels in communication with a center channel.

5. The device of claim 4, wherein said wherein the inner channels are shorter than the outer channels.

6. The device of claim 5, wherein the fluid arrives at the detection area by the inner channels of the microfluidic pathways before the fluid arrives at the detection area by the outer channels of the microfluidic pathways.

7. The device of claim 6, wherein the first rehydrated reagent arrives at the detection area before the second rehydrated reagent.

8. The device of claim 7, wherein the first rehydrated reagent comprises a labeled probe rehydrated from a dried label pad.

9. The device of claim 8, wherein the labeled probe comprises an enzyme label directed to a target analyte, a secondary antibody directed to a target analyte, or an aptamer directed to a target analyte.

10. The device of claim 7, wherein the second rehydrated reagent comprises a substrate rehydrated from a dried substrate pad.

11. The device of claim 10, wherein the substrate comprises a substrate configured to react with the first substrate.

12. The device of claim 1, wherein said detection area comprises a test strip in communication with each of said microfluidic pathways, and wherein each of said test strips are spatially separated.

13. The device of claim 12, wherein each of the test strips comprise a different capture probe configured to bind to a unique target analyte.

14. The device of claim 12, wherein said test strips are in communication with a passive pump configured to generate capillary-driven flow through the each of said microfluidic pathways.

15. The device of claim 1, wherein the fluid arrives at the detection area before the first and second rehydrated reagents.

16. (canceled)

17. The device of claim 1, further comprising a vent in communication with the microfluidic network.

18. The device of claim 1, wherein said air bubble forms adjacent to the fluid inlet.

19. The device of claim 1, wherein said air bubble forms in a central channel positioned between said first and second microfluidic pathways.

20. The device of claim 1, wherein said fluid washes the detection zone prior to said first rehydrated reagent contacting the detection zone.

21-40. (canceled)

41. A multiplex assay device comprising:
a sample inlet;
microfluidic network forming a plurality of microfluidic pathways each extending to a detection area, wherein each of said plurality of microfluidic pathways is in communication with said sample inlet, and further configured to detect a unique target analyte;
a first dried reagent disposed along each of said microfluidic pathways; and
a second dried reagent disposed each of said microfluidic pathways.
